(19) United States
(12) Patent Application Publication
(10) Pub. No.: US 2007/0224669 A1
(43) Pub. Date: Sep. 27, 2007

(54) Self-Pressurizing, Self-Purifying System and Method for Methane Production by Anaerobic Digestion

(75) Inventor: William J. Jewell, Ithaca, NY (US)

Correspondence Address: JONES, TULLAR & COOPER, P.C., P.O. BOX 2266 EADS STATION, ARLINGTON, VA, 22202, US

(73) Assignee: Cornell Research Foundation, Inc., Ithaca, NY (US)

(21) Appl. No.: 11/579,922

(22) PCT Filed: May 13, 2005
(86) PCT No.: PCT/US05/17043

§ 371 Date: Nov. 8, 2006

Related U.S. Application Data
(60) Provisional application No. 60/570,451, filed on May 13, 2004.

(51) Int. Cl. C12P 5/02; C12M 1/107

(52) U.S. Cl. 435/167; 435/290.1

(57) ABSTRACT

Methane is produced using self-pressurizing, self-purifying system (2) and method, which converts a biomass into a biogas using anaerobic digestion. The anaerobic digestion is conducted in a bioreactor (4) that is maintained at a near constant pressure. The biogas that is generated is separated into a non-methane gas and a methane-containing gas. The purified methane-containing gas is stored and/or transported for use as a liquid fuel. The generated methane exhibits an energy density and purity that is equivalent to liquid fuels. The system requires little or no energy input, but is usable to produce methane that is equivalent to conventional liquid fuels in terms of energy density and purity.

### CROSS-REFERENCE TO RELATED APPLICATIONS

This application claims the benefit, under 35 U.S.C. 119(e), of U.S. Provisional Application No. 60/570,451 filed May 13, 2004, the contents of which are incorporated herein by reference.

### BACKGROUND OF THE INVENTION

1. Field of the Invention

The present invention relates, in general, to a system and method for generating a biogas from a biomass. More specifically, the system and method of the present invention is used to generate methane from a biomass using anaerobic digestion.

2. Description of Related Art

Vehicles of all sorts rely upon refined petroleum products, such as gas and motor oil, in order to operate. The increasing number of vehicles built and sold each year ensures that the amount of fuel supplied in a given period of time will eventually not be able to support all the vehicles in operation. Additionally, there are significant and wide-spread concerns about the environmental aspects of fossil fuels attributed significantly to global warming. Fossil fuels are a non-renewable resource having only a finite supply which has sparked concern about energy shortages or a world-wide energy crisis if fossil fuel production ceased or otherwise lagged behind demand. Therefore, alternative energy and fuel research is an important and competitive industry.

Natural gas is one of the cleanest burning fossil fuels, and millions of vehicles worldwide have been modified or built to run on it. In fact, the infrastructure to support the use of natural gas has been developed in some areas where its purer combustion properties are highly valued. Unfortunately, there are a number of drawbacks to using natural gas as a transportation fuel. First, natural gas is still a non-renewable resource. The finite supply of natural gas means the price fluctuates with production. In general, natural gas is not an economically competitive alternative for most consumers. Also, burning natural gas still contributes to global warming gases. Finally, the energy density at which combustion occurs is over one thousand times less than conventional liquid fuels. In order to overcome its low energy density, natural gas must be highly pressurized. High pressures must be combined with low temperatures in order to convert natural gas into a dense, easily transported liquid fuel.

Natural gas mainly consists of methane (CH₄), but, depending on the terrestrial origin of the gas, it can contain other trace gases such as hydrogen sulfide, hydrogen, propane, butane, etc. While natural gas is a non-renewable resource, methane is generated as a natural by-product of anaerobic digestion, which is a ubiquitous environmental process essential for reducing organic matter in the natural environment. The main by-products of anaerobic digestion are methane, at generally one-half to two-thirds of the resulting gas, and carbon dioxide. Almost all of the energy in the original biodegradable organic matter is contained in this renewable source of methane.

One alternative to the heavy reliance on fossil fuels involves purifying the gas that results from anaerobic digestion, also know as “biogas,” in order to produce a pure, renewable methane stream. Typically, anaerobic digestion devices (i.e., anaerobic digestion that is not occurring in nature) are intended to convert organic material, also knows as “biomass,” from one form to another. For example, biomass can be placed in a silo for partial fermentation that converts the biomass to animal feed. Anaerobic digestion is also used to treat plant, animal and human waste. These waste materials can be converted into a fertilizing material. Yet, methane produced from anaerobic digestion would still need to be compressed to greater than 2000 pounds/inch² (2000 ‘psi’) in order to approach the energy density of conventional liquid fuels. Even at 2000 psi, methane is a gas, and it would need to be purified, for some applications, before being used as a fuel. Known biogas purification and compression methods and apparatuses can not produce a cost-effective fuel. As such, methods and devices for producing biogas from anaerobic digestion have been rejected as viable alternatives for the production of fuel. A suitable process would provide a renewable fuel source while treating waste products that must otherwise be disposed of as well as being capable or using most sources of photosynthetically fixed biomass.

The fuel in biogas powered vehicles uses the same engine and vehicle configuration as natural gas vehicles. The gas quality demands are strict. The raw biogas from a digester need to be upgraded in order to obtain biogas which: 1) has a higher calorific value in order to reach longer driving distances; 2) has a regular/constant gas quality to obtain safe driving; 3) does not enhance corrosion due to high levels of hydrogen sulphide, ammonia, and water; 4) does not contain mechanically damaging particles, 5) does not give ice-clogging due to a high water content and 6) has a declared and assured quality. In practice, this means that carbon dioxide, hydrogen sulphide, ammonia, particles and water (and other trace components) have to be removed so that the product gas for vehicle fuel use has methane content above 95%. Different quality specifications for vehicle fuel use of biogas and natural gas are applied in different countries.

A number of biogas upgrading technologies have been developed for the treatment of natural gas, sewage gas, landfill gas, etc. At present, four different methods are used commercially for removal of carbon dioxide from biogas either to reach vehicle fuel standard or to reach natural gas quality for injection to the natural gas grid. These methods include the following: 1) water absorption; 2) polyethylene glycol absorption; 3) carbon molecular sieves; and 4) membrane separation.

Water scrubbing is used to remove carbon dioxide but also hydrogen sulphide from biogas, since these gases are more soluble in water than methane. The absorption process is purely physical. Usually the biogas is pressurized and fed to the bottom of a packed column where water is fed to the top so the absorption process is operated counter-currently. The water which exits the column with absorbed carbon dioxide and/or hydrogen sulphide can be regenerated and recirculated back to the absorption column. The regeneration is made by depressurizing or stripping with air in a similar column. Stripping with air is not recommended when high levels of hydrogen sulphide are handled since the water will soon be contaminated with elementary sulphur which causes operational problems. The most cost efficient method is not to recirculate the water if cheap water can be used, for example, outlet water from a sewage treatment plant.

Polyethylene glycol scrubbing is, like water scrubbing, a physical absorption process. Selexol is one of the trade names used for a solvent. In this solvent, like water, both carbon dioxide and hydrogen sulphide are more soluble than methane. The big difference between water and Selexol is that carbon dioxide and hydrogen sulphide are more soluble in Selexol which results in a lower solvent demand and reduced pumping. In addition, water and halogenated hydrocarbons (contaminants in biogas from landfills) are removed when scrubbing biogas with Selexol. Selexol scrubbing is always designated with recirculation. Due to formation of elementary sulphur, stripping the Selexol solvent with air is not recommended but with steam or inert gas (upgraded biogas or natural gas). Removing hydrogen sulphide beforehand is an alternative.

Molecular sieves are excellent products to separate specifically a number of different gaseous compounds in biogas. Thereby the molecules are usually loosely adsorbed in the cavities of the carbon sieve but not irreversibly bound. The selectivity of adsorption is achieved by different mesh sizes and/or application of different gas pressures. When the pressure is released, the compounds extracted from the biogas are desorbed. The process is therefore often called “pressure swing adsorption” (PSA). To enrich methane from biogas, the molecular sieve is applied, which is produced from coke rich in pores in the micrometer range. The pores are then further reduced by cracking of the hydrocarbons. In order to reduce the energy consumption for gas compression, a series of vessels are linked together. The gas pressure released from one vessel is subsequently used by the others. Usually four vessels in a row are used which are filled with molecular sieves which removes at the same time CO₂ and water vapor.

There are two basic systems of gas purification with membranes: a high pressure gas separation with gas phases on both sides of membrane, and a low-pressure gas liquid absorption separation where a liquid absorbs the molecules diffusing through the membrane. High pressure gas separation needs to pressure gas at 36 bar in a carbon bed to remove H₂S and oil vapor from the compressors. The carbon is followed by a particle filter and a heater. The membranes are made of acetate-cellulose small polar molecules such as carbon dioxide, moisture and the remaining hydrogen sulphide. The raw gas is upgraded in 3 stages to a clean gas with 96% methane or more. The essential element for gas-liquid absorption is a microporous hydrophobic membrane separating the gaseous from the liquid phase. The molecules from the gas stream, flowing in one direction, which are able to diffuse through the membrane will be absorbed on the other side by the liquid flowing in counter current. The absorption membranes work at approximately atmosphere pressure (1 bar) which allows low-cost construction. The removal of gaseous components is very efficient. At a temperature of 25 to 35° C. the H₂S concentration in the raw gas of 2% is reduced to less than 250 ppm, and biogas can be upgraded from 55% to 96% of CH₄. The absorbent is either Coral or NaOH.

Additional work has been done involving in situ methane enrichment in methanogenic energy crop digesters. This system was designed to recycled CO₂-rich leachate from the digester to an external gas-stripping column. Digester offgas is accumulated in a bag, and a screened intake manifold in the bottom of the digester allows liquid in which CO₂ dissolved to drain from the digester and flow into the gas stripper. The open top of the stripper allowed the sweep gas and CO₂ to be vented to the atmosphere. Operation of this simple ambient pressure digester system utilizing leachate recycle to an external stripper can achieve high-quality CH₄. However, it has a number of limitations, in particular, the removal of CO₂ in the external stripper caused the pH to increase substantially so the liquid that was recycled back to the digester had a high pH.

In general, the final production of purified methane through all of the above methods is with normal atmosphere pressure. If the methane is used as a vehicle fuel to replace compressed natural gas, additional energy will eventually be needed to compress the methane to the required high pressure.

Another crucial point for various biogas upgrading technologies is their associated costs. The cost for upgrading biogas, from small scale anaerobic digesters, using existing technologies could be very high. The cost concerns associated with most of the existing technologies is time and the physical solvents used. The principal advantages of water as an absorbent are its availability and low cost.

Therefore, there exists a need for a self-pressurizing, self-purifying system for methane production using anaerobic digestion. Presently available biogas systems are not economical and cannot produce sufficiently pressurized or pure methane.

### BRIEF SUMMARY OF THE INVENTION

In accordance with the present invention, a self-pressurizing, self-purifying system and method for producing methane by anaerobic digestion is provided. The system of the present invention requires little or no energy input, but is usable to produce methane that is equivalent to conventional liquid fuels in terms of energy density and purity. The present invention overcomes the limitations of the prior devices, and is a substantial advance in the art.

Generally, the preferred self-pressurizing, self-purifying system of the present invention comprises two modules: a self-pressurizing bioreactor and a self-purifying tank. In use, a feed chamber is filled with a feed material including a quantity of biomass preferably saturated in water. The feed chamber can be pressurized or it can operate at ambient pressures. A positive displacement feed apparatus preferably moves the feed material from the feed chamber to a bioreactor. As the biomass is added to the bioreactor previously digested material is preferably withdrawn from the bioreactor. Biomass addition and digested material extraction from the bioreactor are preferably accomplished under equal pressure thus eliminating any compressing energy. Preferably, the feed chamber and an effluent container are then reduced to ambient pressure and effluent is expelled to the effluent container. The digested material is preferably removed from the system for further processing or recycling.

The bioreactor contains a volume of biomass that is subject to anaerobic digestion. The system acts to maintain a nearly constant pressure within the bioreactor. The digestion reaction creates a gas by-product known as a “biogas”. The biogas exits the bioreactor via a biogas pipe and enters the self-purifying tank which preferably contains a volume of stripping liquid. Preferably, the pressurized biogas percolates through the stripping liquid. Non-methane gases that are soluble within the stripping fluid are preferably absorbed and, thus are removed from the biogas. The remaining methane-containing gas that is not absorbed, consisting mainly, if not completely, of methane, exits the self-purifying tank. The resulting pressurized and purified methane is preferably transferred to mobile storage containers or a pipeline.

The effectiveness of the method and system of the present invention is generally based on three principles. First, high pressure has little or no impact on metabolic activities in a microbial system. In fact, reactors in anaerobic digestion laboratories have been known to explode as anaerobic methane fermentation continues even as pressure in the reactors builds. On the other hand, rapid changes in pressure can have a lethal effect on a methane fermentation system. Therefore, the system of the present invention is constructed to maintain a constant pressure in the bioreactor. Bacteria used for the anaerobic digestion is viable until the soluble concentration of by-products begins to influence other environmentally sensitive factors such as the pH of the microbes or the bulk solution. Methane is a neutral chemical so it has little or no impact on microbial metabolism. Carbon dioxide production can be buffered so that the carbon dioxide will not depress the system's pH level. The end effect is that gas produced by the anaerobic digestion is automatically pressurized by the bacterial metabolic activity. The second principle relates to the incompressible nature of water. Water can be used in the feed chamber so that little or no work is required to feed or expel material within system. If the bioreactor was operated at 2000 psi, high energy inputs would be required to feed the organic substrate and withdraw the digested material since it would be necessary to push the material from an ambient one atmospheric pressure to the pressure found in the bioreactor. Instead, the biomass is saturated with water or another incompressible fluid. The saturated biomass is either pressurized within the feed chamber or left at ambient pressure. A transfer pump and/or the positive displacement pump transfers the biomass to the bioreactor via a set of fluid connectors and valves. The bacterial metabolic activity produces the biogas that self-pressurizes the sealed bioreactor. The final principle in use relies upon the fact that different gases have different solubilities in liquid. For the present invention, this means that when the pressurized biogas is injected into a fluid filled tank, some gases will be dissolved or absorbed by the fluid while others will not. Specifically, carbon dioxide and other gases will be trapped in the fluid tank while methane, still under pressure, will pass to a storage tank or pipeline. Purification could also be achieved via other know purification or filtration methods.

The self-pressurizing, self-purifying system and method of the present invention overcomes the limitations that prevented such systems from being viable alternative fuel sources. The present invention creates a renewable energy source that produces high density fuel sources. The present invention creates a renewable energy source that produces a fuel that is cleaner than conventional fossil fuels. The methane produced by the present invention, however, has nearly equal, if not greater, energy density and purity in comparison to convention fluid fuels.

### BRIEF DESCRIPTION OF THE DRAWINGS

The features and advantages of the present invention will become apparent from the following detailed description of a preferred embodiment thereof, taken in conjunction with the accompanying drawings, in which:

FIG. 1 is a schematic depicting the preferred system 2 of the present invention including a self-pressurizing bioreactor 4 and a self-purifying tank 6;

FIG. 2 is a schematic depicting the system 2 including two preferred high pressure two-way valves 80, 82 in connection with the bioreactor 4;

FIG. 3 is a schematic depicting the system of FIG. 2 with valves 80, 82 reversed and all lines and the pump 14 filled with raw feed material 10 including biomass;

FIG. 4 is a schematic depicting the system 2 including two preferred high pressure two-way valves 90, 92 in connection with the self-purifying tank 6;

FIG. 5 is a schematic depicting the system 2 of FIG. 4 with valves 90, 92 reversed;

FIG. 6 is a schematic depicting the system 2 of FIG. 5 with valves 90, 92 once again reversed to place the self-purifying tank 6 under pressure.

### DETAILED DESCRIPTION OF THE INVENTION

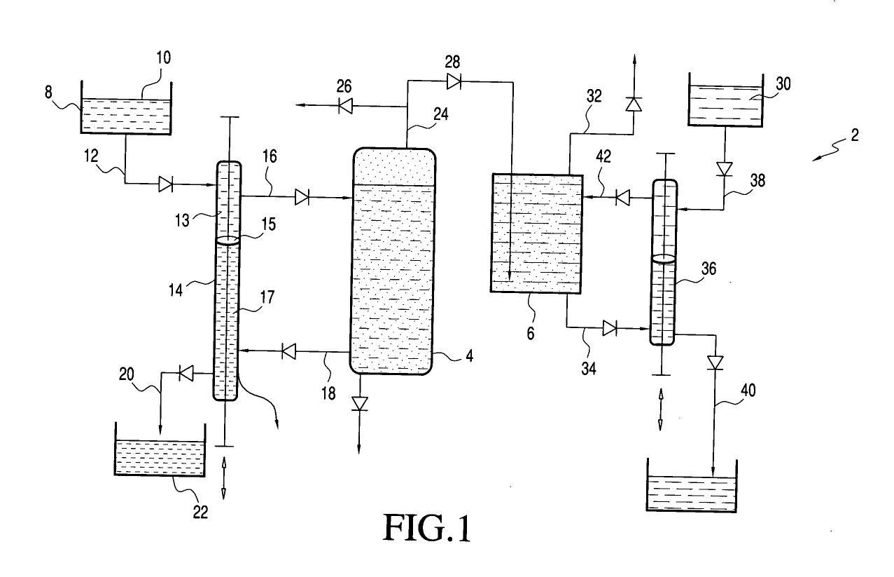

FIG. 1 illustrates a first preferred embodiment of a self-pressurizing, self-purifying system 2 in accordance with the present invention. System 2 preferably includes a self-pressurizing bioreactor 4 and a self-purifying tank 6. A feed chamber 8 preferably holds a feed material 10 including biomass, preferably saturated with water or some other incompressible fluid. The biomass is selected from a variety of known organic materials, including, but not limited to manure, crop/wood residue, food waster, and wheat straw. Preferably, the feed material 10 includes biomass and at least 75% water as a fraction of the wet weight of the feed material 10. Feed chamber 8 may be pressurized or maintained at ambient pressure. Feed material 10 is preferably drawn through a feed pipe 12 by a positive displacement feed apparatus, such as a positive displacement pump 14. The positive displacement pump 14 provides a “space lock” or “pressure lock” for the system 2. Pump 14 preferably includes a plunger 15 which drives the feed material 10 through a feed reactor pipe 16 to bioreactor 4. Microbes in bioreactor 4 anaerobically digest the biomass in the feed material 10 producing digested material and a biogas. The biogas includes methane gas. The bioreactor 4 is naturally pressurized by the biogas that is generated during the anaerobic digestion reaction. An active methanogenic microbial ecosystem preferably converts biodegradable organic matter in the biomass to biogas in the bioreactor 4. The digesting material is preferably removed through the digested material pipe 18 and replaces a volume of feed material 10 in the pump 14. The pump 14 preferably maintains a constant fluid/feed volume in bioreactor 4 by withdrawing a volume of digested material from the bioreactor 4 that is equivalent to the volume of feed material 10 that is added to the bioreactor 4. The digested material that is withdrawn from the bioreactor 4 is pushed by pump 14 through an effluent outlet pipe 20. The digested material is preferably expelled at ambient pressure into an effluent chamber 22 where it can be processed further or recycled.

A series of one-way valves along with a pressure locked pump 14 and the plunger 15 preferably maintains a fixed pressure in the preferred bioreactor 4. The plunger 15 divides the pump 14 into a first chamber 13 and a second chamber 17. The two chambers can be varied by forcing the plunger 15 through the first chamber 13 thereby discharging the contents in the second chamber 17 while simultaneously filling the first chamber 13. As long as the pressures in both chambers are equal, moving the plunger 15 is relatively effortless. The plunger 15 preferably includes an O-ring type disk that sufficiently fits within the pump 14 to equalize pressure. Leakage from the first chamber 13 to the second chamber 17 is insignificant as long as the pressures are equal. The energy required to move the plunger 15 remains insignificant in that the only force needed is the force necessary to overcome liquid friction pressure in the lines and the friction of the O-ring against the side of the chambers. The feed reactor pipe 16 and the digested material pipe 18 are preferably high-pressure lines that are open when feed material 10 is transferred to the bioreactor 4 or digested material is removed from the bioreactor 4. The feed pipe 12 and the effluent outlet pipe 20 are preferably low pressure lines. The pump 14 generally acts in batch cycling mode.

Returning to FIG. 1, self-compressed biogas is controllably released from the top of the bioreactor 4 through biogas pipe 24. The biogas pipe 24 preferably includes a safety relief valve 26 and a one-way biogas relief valve 28. The biogas pipe 24 feeds pressurized biogas to the self-purifying tank 6. Preferably, the biogas is fed to the bottom of the self-purifying tank 6 which is filled with a stripping liquid 30, preferably water. Self-purifying tank 6 is preferably maintained at a pressure less than the bioreactor 4 thereby enabling the biogas to be processed with minimum transfer energy. The pressure within the self-purifying tank 6 is preferably at least 1000 psi. Preferably, the biogas percolates through the stripping liquid 30 and a non-methane gas including impurities, such as carbon dioxide, is preferentially absorbed by the stripping liquid 30. An unabsorbed biogas, referred to herein as a methane-containing gas, including mainly or entirely methane gas, exits the self-purifying tank 6 via a methane outlet 32. Preferably, the methane-containing gas exiting the methane outlet 32 includes at least 90% methane gas, most preferably at least 95% methane gas. The purified methane gas is then preferably stored in mobile storage tanks or sent to a pipeline.

In one preferred embodiment, a stripping fluid outlet 34 circulates stripping liquid 30, which has absorbed impurities from the biogas, through a gas stripper device 36. The preferred embodiment illustrated in FIG. 1 shows a positive displacement pump as the gas stripper device 36 similar to the pressure lock pump 14 previously described. The gas stripper device 36 includes a stripper liquid feed 38 and an unused liquid outlet 40. Stripper liquid from the gas stripper device 36 is fed to the self-purifying tank 6 via stripper recycling line 42. A person skilled in the art will appreciate that other gas stripper devices could be used to purify the stripping liquid. The illustrated embodiment of the self-purifying tank 6 provides for continuous purification within a closed system. The self-purifying tank 6 may include a gas transfer or mixing device such as a self-aspirating aerator or mixer to assist in transferring gas to the liquid. Once processed, stripping liquid from the unused liquid outlet 40 could be returned to the stripper liquid feed 38 for repeat absorption of gases.

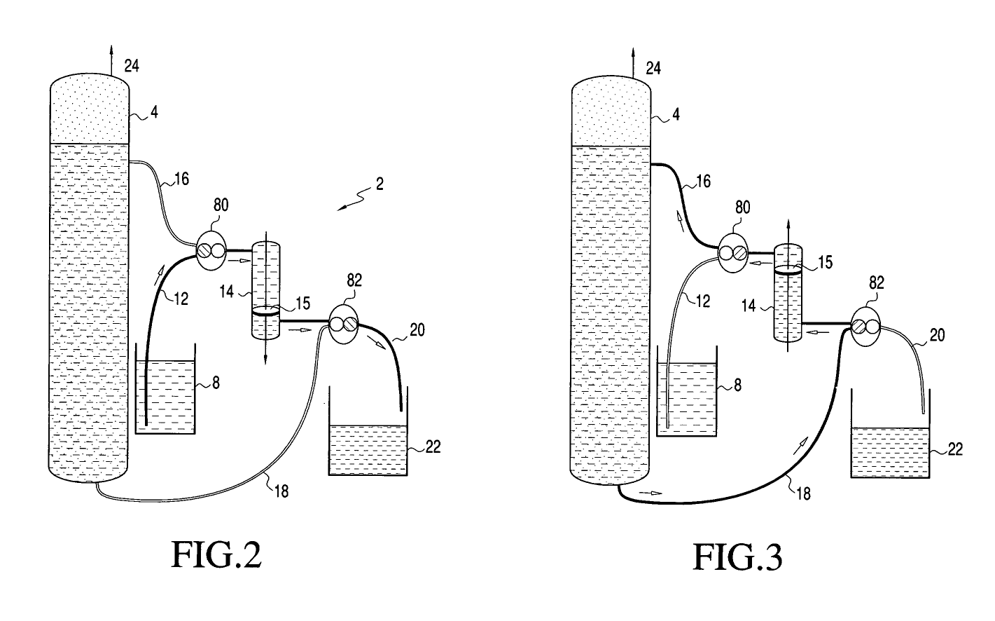

In an alternate preferred embodiment shown in FIG. 2, the system 2 may include two high pressure two-way valves 80, 82 in connection with the feed chamber 8, the bioreactor 4, the pump 14 and the effluent container 22. In the following description, the pump 14 has just completed high-pressure transfer of the feed material 10 and is being emptied at ambient pressure. In preparation for feeding new feed material 10 to the bioreactor 4 and emptying the digested material from the pump 14, valve 80 is closed to the bioreactor and opened to the feed pipe 12. Valve 82 is closed to the bioreactor 4 and opened to the effluent container 22. All pipes and chambers are preferably at zero psig. The plunger 15 has been depressed downward and the pump 14 is now filled with new feed material 10. Simultaneously, the digested material of the pump 14 is preferably discharged into the effluent container 22 at ambient pressure. As shown in FIG. 3, valves 80, 82 are reversed and all lines and the pump 14 are filled with raw feed material 10 following the transfer. All pressures are now at the preferred bioreactor pressure of at least 1,000 psi. After reversing the valves 80, 82 and the plunger 15, the raw feed material 10 is added to the top of the bioreactor 4 and an equal amount of the digested material is sucked from the bottom of the bioreactor 4 into the now pressurized pump 14. Now the bioreactor 4 is filled with digested material and the position of valves 80, 82 are reversed in preparation for transferring the digested material to the effluent container 22 and sucking up an equal volume of feed material 10 from the feed chamber 8 at ambient pressure into the pump 14. This cycling can be frequent and enable the bioreactor 4 to approach a continuously flowing system, or it could occur infrequently, say once per week.

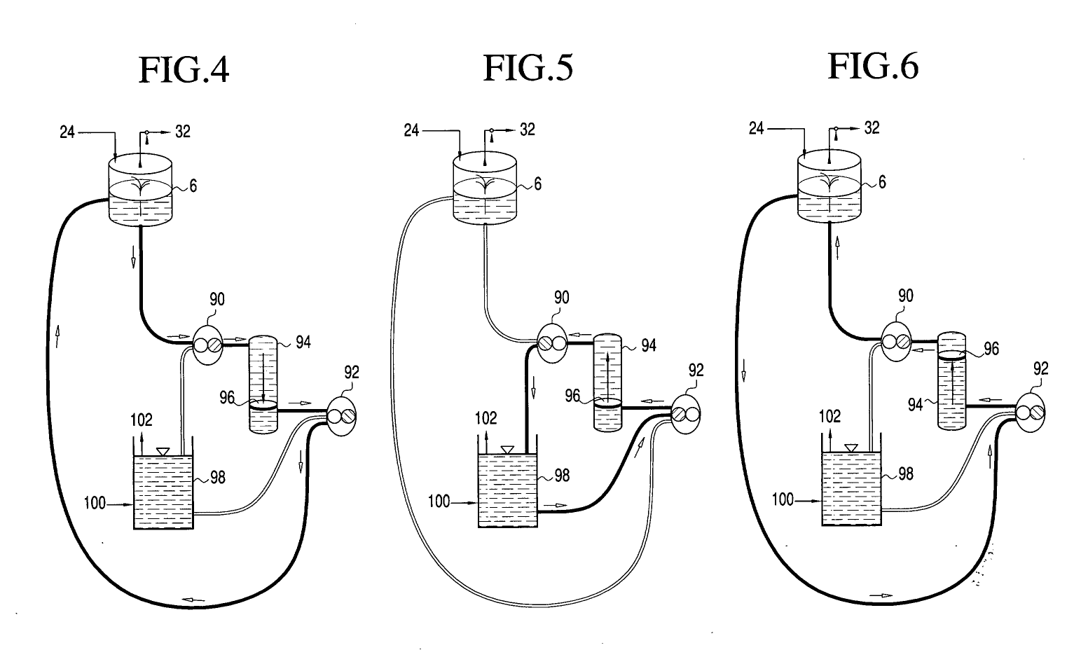

In an alternate preferred embodiment shown in FIG. 4, the system 2 may include two high pressure two-way valves 90, 92 in connection with the self-purifying tank 6. Valves 90, 92 are open to the self-purifying tank 6 that receives biogas from the bioreactor 4. Previous to the situation shown in FIG. 4, liquid that had been stripped of the target gases filled a transfer vessel 94. A plunger 96 is depressed downward and the degassed liquid is returned to the self-purifying tank 6 so that it can take up additional gas. A batch of stripping liquid containing large quantities of methane and carbon dioxide are sucked into the transfer chamber 94. All lines shown as bold in FIG. 4 are at bioreactor pressures, preferably at least 1000 psi. In preparation for transferring the gas saturated liquid to a stripping unit 98 that is at ambient pressure, valves 90, 92 are reversed, and this opens the system to atmospheric pressure as shown in FIG. 5. The plunger 96 is raised thus depositing the saturated liquid into the stripping unit 98 in readiness to transfer this volume of gas stripped liquid back to the self-purifying tank 6. Once the transfer is complete, the valves 90, 92 are once again reversed thus placing the self-purifying tank 6 under pressure, preferably at least 1000 psi, as shown in FIG. 6. Raising the plunger 37 deposits the stripped liquid back in the self-purifying tank 6 to take up another batch of gases, while a near saturated volume of liquid is transferred to the transfer chamber 94 in preparation for gas manipulation at ambient pressure. The stripping unit 98 preferably includes a stripping gas inlet 100 and a stripped gas outlet 102. The stripped gas could be recovered at varying purity in the stripper 98 by adding multiple chambers.

It will be understood that alternative constructions are available. Notably, the positive displacement feed apparatus 14 is not limited to positive displacement pumps utilizing plungers. Similarly, the self-purifying tank 6 could be in the form of a membrane or filter that separates methane from other gases found in the biogas.

Although the present invention has been disclosed in terms of a preferred embodiment, it will be understood that numerous additional modifications and variations could be made thereto without departing from the scope of the invention as defined by the following claims:

What is claimed is:

1. A self-pressurizing, self-purifying system for producing methane by anaerobic digestion comprising:
a.) a feed chamber for supplying a feed material including a biomass to the system;
b.) a positive displacement feed apparatus connected to said feed chamber;
c.) a self-pressurizing bioreactor connected to said positive displacement feed apparatus, said bioreactor including a plurality of microbes for converting the biomass to a digested material and a biogas, said biogas including methane; and
d.) a self-purifying tank connected to said bioreactor; said self-purifying tank receives said biogas from said bioreactor at a pressure above ambient pressure and provides for the separation of said biogas into a non-methane gas and a methane-containing gas.

2. The system of claim 1, wherein said positive displacement feed apparatus is a positive displacement pump having a first chamber and a second chamber separated by a plunger.

3. The system of claim 1, wherein said self-purifying tank includes a stripper liquid.

4. The system of claim 3, wherein said stripper liquid is water.

5. The system of claim 3, further comprising
e) a transfer vessel connected to said self-purifying tank.

6. The system of claim 5, further comprising
f) a stripping unit connected to said transfer vessel for supplying the stripping liquid to the system.

7. The system of claim 1, wherein said bioreactor is maintained at a fixed pressure above ambient pressure.

8. The system of claim 7, wherein said bioreactor is maintained at a fixed pressure of at least 1000 psig.

9. The system of claim 1, further comprising
e) an effluent container connected to said bioreactor for receiving said digested material.

10. The system of claim 1, wherein said positive displacement feed apparatus is connected to said feed chamber through a feed pipe and connected to said bioreactor through a positive displacement reactor pipe and a digested material pipe.

11. The system of claim 10, wherein said feed material including said biomass is added to the bioreactor through the reactor pipe simultaneously with the removal of the digested material through the digested material pipe.

12. The system of claim 1, wherein said feed material further includes water.

13. The system of claim 12, wherein said feed material includes at least 75% water as a fraction of the weight percent of said feed material.

14. The system of claim 1, wherein said self-purifying tank maintains a pressure of at least 1000 psig.

15. A method for producing methane by anaerobic digestion comprising:
a) generating a biogas and a digested material by subjecting a feed material including a biomass to anaerobic digestion within a self-pressurizing bioreactor, said biogas includes methane;
b) continuing the generation of biogas until the self-pressurizing bioreactor reaches a fixed pressure above ambient pressure;
c) removing said biogas from the self-pressurizing bioreactor once the fixed pressure is reached; and
d) separating said biogas that is removed from the self-pressurizing bioreactor into a non-methane gas and a methane-containing gas.

16. The method of claim 15, wherein said fixed pressure of step b) is at least 1000 psig.

17. The method of claim 15, wherein said step of separating methane from said biogas includes using a stripping liquid.

18. The method of claim 17, wherein said stripping liquid is water.

19. The method of claim 15, further comprising adding the feed material including the biomass to the self-pressurizing bioreactor simultaneously with a removal of the digested material from the self-pressurizing bioreactor.

20. The method of claim 15, wherein said feed material further includes water.

21. The method of claim 20, wherein said feed material includes at least 75% water as a fraction of the weight percent of said feed material.

22. The method of claim 15, wherein said methane-containing gas includes at least 90% methane.

23. The method of claim 15, wherein said step of separating said biogas is maintained at a pressure of at least 1000 psig.

24. The method of claim 15, further comprising introducing said feed material to said self-pressurizing bioreactor through a positive displacement feed apparatus.

25. The method of claim 24, further comprising removing said digested material from said self-pressurizing bioreactor through said positive displacement feed apparatus.
